(19) United States
(12) Patent Application Publication
(10) Pub. No.: US 2023/0406835 A1
(43) Pub. Date: Dec. 21, 2023

(54) WASHING AND EXTRACTION OF CANNABINOIDS

(71) Applicant: CHEMTOR, LP, Lockhart, TX (US)

(72) Inventor: William LANIER, West Jordan, UT (US)

(21) Appl. No.: 18/328,062

(22) Filed: Jun. 2, 2023

Related U.S. Application Data
(63) Continuation of application No. PCT/US2023/022672, filed on May 18, 2023.
(60) Provisional application No. 63/364,922, filed on May 18, 2022.

(51) Int. Cl. C07D 311/80; C07C 37/80; B01D 11/04

(52) U.S. Cl. CPC C07D 311/80; C07C 2601/16; B01D 11/0492; C07C 37/80

(57) ABSTRACT

A method of remediating cannabinoid containing compositions includes contacting the compounds with an aqueous solution to remove at least one impurity therefrom. The method may include additional contacting with further aqueous solutions of the same or different kind in order to remove additional impurities or higher amounts of the same impurity. A system for remediating cannabinoid containing compositions includes a cannabinoid containing composition supply and an aqueous solution supply fluidly coupled to a conduit reactor and a separator for collecting reaction products between the compound and the aqueous solution.

### CROSS REFERENCE TO RELATED APPLICATIONS

The present application is a continuation of PCT International Patent Application No. PCT/US2023/022672 filed May 18, 2023, and entitled “WASHING AND EXTRACTION OF CANNABINOIDS,” which claims benefit of priority to U.S. Provisional Patent Application No. 63/364,922 filed May 18, 2022, entitled “WASHING AND EXTRACTION OF CANNABINOIDS,” the entirety of which are hereby incorporated by reference.

### FIELD OF DISCLOSURE

The present disclosure is related to purification and extraction of cannabinoids. More particularly, this disclosure is related to methods and systems of washing and/or extracting cannabinoids in feedstock oils, such as crude cannabis extracts, distillates, and synthetic conversions. The disclosure is further related to methods and systems for converting cannabinoids within said feedstock oils.

### BACKGROUND

Cannabinoids occur in the hemp plant, Cannabis sativa, primarily in the form of cannabinoid carboxylic acids (referred to herein as “cannabinoid acids”). The more abundant forms of cannabinoid acids include tetrahydrocannabinolic acid (THCA), cannabidiolic acid (CBDA), cannabigerolic acid (CB GA) and cannabichromic acid (CBCA). Other acid cannabinoids include, but are not limited to, tetrahydrocannabivaric acid (THCVA), cannabidivaric acid (CBDVA), cannabigerovaric acid (CBGVA) and cannabichromevaric acid (CBCVA). “Neutral cannabinoids” are derived by decarboxylation of their corresponding cannabinoid acids. The more abundant forms of neutral cannabinoids include tetrahydrocannabinol (THC), cannabidiol (CBD), cannabigerol (CBG) and cannabichromene (CBC). Other neutral cannabinoids include, but are not limited to, tetrahydrocannabivarin (THCV), cannabidivarin (CBDV), cannabigerovarin (CBGV), cannabichromevarin (CBCV) and cannabivarin (CBV).

Cannabinoids may be extracted from the hemp plant using organic solvents. The crude extracts contain many unwanted contaminants, such as pesticides, heavy metals, gums, colorants, and odor causing substances. Removing these contaminants can be labor intensive and expensive, often requiring large volumes of solvent and five or more washes. Even after purifying the crude extract using existing methods to yield, e.g., a cannabinoid distillate, some unwanted components may still remain, which leads to a less desirable product.

Synthetic cannabinoids have also been made using more abundant (or more easily isolated) cannabinoids or terpenes as a starting material to yield a different cannabinoid or cannabinoid acid (i.e., a synthetic cannabinoid). Many cannabinoids are known to cause very distinct physiological reactions in humans (e.g., for use in inflammation treatment), making isolates of several species very desirable. However, some cannabinoids are difficult to isolate and/or are minor species within the hemp plant, therefore raising the need for the aforementioned synthetic conversions.

For example, CBD may be synthetically converted into THC (e.g., 48 or 49). Another example is converting CBD to CBDA, wherein CBDA has been recently discovered to bind to viral proteins to block infection. However, these conversion reactions have several limitations. For instance, the reactions involve dangerous reagents that are unsafe for human consumption. As such, the synthetic cannabinoids must be carefully and thoroughly washed of these chemicals before yielding a marketable product. Moreover, these reactions are rarely complete, with most or all yielding only about a 50/50 blend of the starting material and the desired cannabinoid. Generally, it is impractical to then separate the components, for instance, via chromatography. As such, there is a need for a remediation system that can remove impurities and/or separate synthetic cannabinoids from the unconverted precursor cannabinoids.

Another issue with cannabinoid extracts and synthetic cannabinoids is the highly variable nature of the components within the hemp plant. That is, each crop and even plants within the same crop may have varying amounts of desirable and undesirable components. In view of these numerous issues, there remains a need for a remediation system that is capable of accommodating a wide variety of compositions.

### BRIEF DESCRIPTION OF THE DRAWINGS

The following FIGURES illustrate embodiments of the subject matter disclosed herein. The claimed subject matter may be understood by reference to the following description taken in conjunction with the accompanying FIGURES, in which:

FIG. 1 is a schematic diagram of a system according to embodiments of the present disclosure.

FIG. 2 is a process diagram of a method according to embodiments of the present disclosure.

FIG. 3 is a schematic diagram of a system according to embodiments of the present disclosure.

FIG. 4 is a process diagram of a method according to embodiments of the present disclosure.

### DETAILED DESCRIPTION

The following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

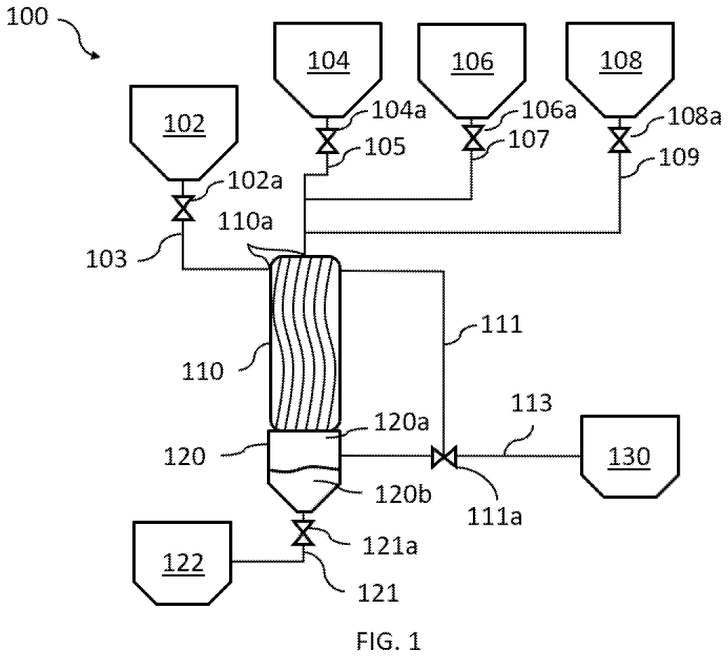

Referring to FIG. 1, a system 100 is shown for washing and/or extracting components (also referred to herein as impurities) from a cannabinoid containing composition or for converting cannabinoids. The cannabinoid containing composition is supplied into the system via cannabinoid supply 102. In some embodiments, the cannabinoid containing composition is a crude extract, a cannabinoid distillate, or a synthetic cannabinoid reaction. In some embodiments, the cannabinoid containing composition may include an emulsion or suspension of organic particles (solid or liquid) in an aqueous phase. For example, bubble hash is formed by separating trichomes from the hemp plant using cold water. The wastewater from this process includes cannabinoids and may be used herein as the cannabinoid containing composition. In such embodiments, the aqueous phase may have an organic solvent added thereto prior to processing in order to better extract and isolate the cannabinoids.

The cannabinoid containing composition includes at least one cannabinoid or cannabinoid acid (referred to herein collectively as “cannabinoids”). For example, the cannabinoid containing composition may include tetrahydrocannabinolic acid (THCA), cannabidiolic acid (CBDA), cannabigerolic acid (CBGA), cannabichromic acid (CBCA), tetrahydrocannabivaric acid (THCVA), cannabidivaric acid (CBDVA), cannabigerovaric acid (CBGVA), cannabichromevaric acid (CBCVA), tetrahydrocannabinol (THC), cannabidiol (CBD), cannabigerol (CBG) and cannabichromene (CBC), tetrahydrocannabivarin (THCV), cannabidivarin (CBDV), cannabigerovarin (CBGV), cannabichromevarin (CBCV), cannabivarin (CBV), or combinations thereof. The cannabinoid containing composition may also contain one or more types of terpenes, flavonoids, and/or carotenoids. In some embodiments, the cannabinoid containing composition includes an organic solvent into which the cannabinoids are dissolved (an organic phase). In some embodiments, the organic solvent is a hydrocarbon. For example, the organic solvent can include heptane, butane, propane, hexane, olive oil, methyl chloride, petroleum ether, methyl tert-butyl ether, or combinations thereof. The cannabinoid containing composition may also include some water content in the form of an aqueous phase and/or an emulsion.

The cannabinoid containing composition includes at least one impurity. In some embodiments, the at least one impurity includes free fatty acids (FFAs), pesticides, gums, phospholipids, colorants or pigments, odor causing substances, acidic cannabinoids, sugars, cellulose, hemicellulose, chlorophyl, other contaminants, or combinations thereof. In some embodiments, the impurity comprises a polar solvent, such as an alcohol, ethyl acetate, or acetone. In this regard, polar solvents may be used to extract cannabinoids from the cannabis plant (biomass) wherein said solvents can constitute an impurity in the extract that needs to be removed. For example, cannabinoids may be extracted from biomass using cryo-ethanol and the resultant extract (cannabinoid containing composition) may include residual ethanol.

The cannabinoid supply 102 is in communication with a first conduit reactor 110 via line 103. Line 103 may include a valve 102a positioned between the cannabinoid supply 102 and the first conduit reactor 110 to control a flow of the cannabinoid containing composition to the inlet 110a of the first conduit reactor 110. Although a plurality of inlets 110a are depicts, in some embodiments, the first conduit reactor includes a single inlet 110a. The conduit reactor may be a fiber reactor as described in U.S. Pat. No. 11,198,107, which is hereby incorporated by reference in its entirety. In the system 100, the cannabinoid containing composition is simultaneously introduced into the first conduit reactor 110 with an aqueous solution. The system includes at least two aqueous supplies fluidly connected to the first conduit reactor 110 and configured to supply an aqueous solution thereto. In FIG. 1, a first aqueous supply 104, a second aqueous supply 106, and a third aqueous supply 108 are shown.

The first aqueous supply 104 is in communication with the first conduit reactor 110 via line 105. Line 105 may include a valve 104a positioned between the first aqueous supply 104 and the first conduit reactor 110 in order to control a flow of aqueous solution to the inlet 110a of the first conduit reactor 110. In some embodiments, the first aqueous supply 104 includes an acidic solution having a pH of less than 7. In some embodiments, the acidic solution comprises hydrochloric acid, phosphoric acid, nitric acid, sulfuric acid, citric acid, other food-safe acids, mineral acids, or combinations thereof. In some embodiments, the acidic solution further comprises a salt dissolved therein, such as sodium chloride, potassium chloride, sodium iodide, and the like. In some embodiments, the acidic solution has a pH of greater than 3.5 to less than 7, greater than 3.5 to 6.5, or greater than 4 to less than 6. If the acidic solution is too acidic, it may undesirably react with components in the cannabinoid containing composition and additional undesired reactions and/or conversions can take place.

The second aqueous supply 106 is in communication with the first conduit reactor 110 via line 107. Line 107 may include a valve 106a positioned between the second aqueous supply 106 and the first conduit reactor 110 in order to control a flow of aqueous solution to the inlet 110a of the first conduit reactor 110. In some embodiments, the second aqueous supply 106 includes a basic solution having a pH of greater than 7. In some embodiments, the basic solution comprises sodium hydroxide, potassium hydroxide, lithium hydroxide, sodium bicarbonate, sodium carbonate, calcium hydroxide, ammonia, other food-safe bases, or combinations thereof. In some embodiments, the basic solution further comprises a salt dissolved therein, such as sodium chloride, potassium chloride, sodium iodide, and the like. In some embodiments, the basic solution has a pH of greater than 7 to less than 13, greater than 8 to 12.5, or greater than 8 to less than 12. If the basic solution is too basic, it may undesirably react with components in the cannabinoid containing composition.

The third aqueous supply 108 is in communication with the first conduit reactor 110 via line 109. Line 109 may include a valve 108a positioned between the third aqueous supply 108 and the first conduit reactor 110 in order to control a flow of aqueous solution to the first conduit reactor 110. In some embodiments, the first aqueous supply 104 includes water or a brine. In some embodiments, the water or brine has a pH of approximately 7, 6 to 8, or 6.5 to 7.5. In some embodiments, the brine includes a salt dissolved therein, such as sodium chloride, potassium chloride, sodium iodide, and the like. In some embodiments, the brine comprises greater than 0 wt % to 20 wt %, 1 wt % to 15 wt %, or 2 wt % to 10 wt % of dissolved solids. If the brine is too saturated, it may not effectively extract impurities from the cannabinoid containing composition.

In any embodiment, the first, second, and/or third aqueous supplies 104, 106, 108 (collectively “aqueous supplies”) may include any of the aforementioned acids, bases, and/or brines. In some embodiments, one or more the aqueous supplies may include an additive. For example, a chelating agent and/or a phase transfer catalyst may be added to one or more the aqueous supplies. Examples of phase transfer catalysts include, but are not limited to, quaternary ammonium salts, such as benzyltriethylammonium chloride, methyltricaprylammonium chloride and methyltributylammonium chloride, phosphonium salts, or combinations thereof. Examples of chelating agents include, but are not limited to, ethylenediaminetetraacetic acid (EDTA), disodium tartrate dihydrate (DTD), or trisodium citrate dihydrate (TCD), oxalic acid, or combinations thereof. Such additives may target components of the cannabinoid containing composition that are otherwise difficult to extract into an aqueous solution. In some embodiments, the additive may be added directly to the cannabinoid containing composition instead of, or in addition to, being added to one or more of the aqueous supplies. In some embodiments, the additive may include an alcohol.

The first conduit reactor 110 includes a separator 120 at an end opposite of the inlets 110a. In operation, the first conduit reactor 110 processes an organic liquid (e.g., the cannabinoid containing composition) and an aqueous liquid (e.g., the aqueous solution from supplies 104, 106, and/or 108) and produces two separate phases: a lighter organic phase 120a and a heavier aqueous phase 120b, which form separate layers in the separator 120. In some embodiments, a third layer may be present between the organic phase 120a and the aqueous phase 102b, i.e., an emulsion layer. In some embodiments, the emulsion layer is removed from the separator 120 with the organic phase 120a. In other embodiments, the emulsion layer is removed from the separator 120 with the aqueous phase 120b. In yet other embodiments, the emulsion layer may be removed separately from the organic phase 120a and the aqueous phase 120b.

The system 100 includes a line 121 for removing the aqueous phase 120b (and optionally an emulsion layer) from the separator 120. Line 121 may include a valve 121a for controlling flow out of the separator 120. The line 121 is fluidly connected to an effluent tank 122 that is configured to collect the spent aqueous solution. The spent aqueous solution includes the aqueous solution from supplies 104, 106, and/or 108 and at least one impurity from the cannabinoid containing composition (or from the organic phase reintroduced into the first conduit reactor 110). The at least one impurity in each of the spent aqueous solutions may be the same or different. In some embodiments, at least two of the spent aqueous solutions include impurities from the cannabinoid containing composition that are different in kind (i.e., different compounds). In some embodiments, at least two of the spent aqueous solutions include the same type of impurity but at different concentrations (e.g., one of the aqueous solutions may extract a greater or lesser amount of the same impurity from the cannabinoid containing composition that another of the aqueous solutions). The spent aqueous solution may be recycled into the system 100, processed to isolate desirable compounds, remediated to, e.g., neutralize the spent aqueous solution prior to discarding the same, and/or discarded. Portions of the spent aqueous solution may be separately processed in any of the foregoing manners.

The system 100 further includes a line 111 for directing the organic phase 120a (and optionally an emulsion layer) back into the inlet 110a of the first conduit reactor 110. Line 111 may also connect to a line 113 for directing a final product to a cannabinoid oil tank 130. The line 111 may include one or more valves 111a for controlling flow of the organic phase 120b out of the separator 120 and/or controlling flow into the cannabinoid oil tank 130.

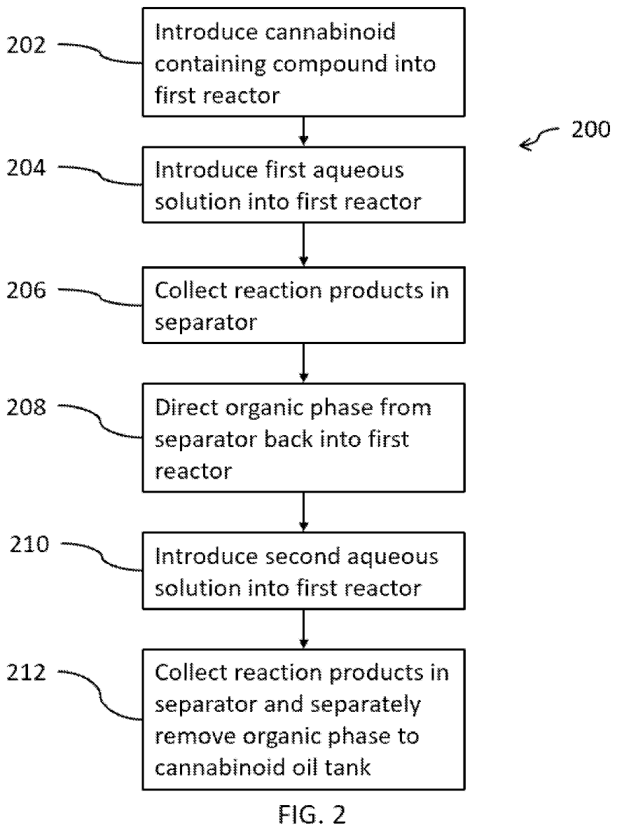

Turning to FIG. 2, a method 200 of operating the system 100 is depicted. In step 202, the cannabinoid containing composition is supplied into an inlet 110a of the first conduit reactor 110 via line 103. In step 204, which may be conducted simultaneously with step 202, a first aqueous solution is supplied to an inlet 110a of the first conduit reactor 110 from one or more of the first aqueous supply 104, the second aqueous supply 106, and the third aqueous supply 108 via lines 105, 107, and 109, respectively. In step 206, the cannabinoid containing composition and aqueous solution are contacted with one another in the first conduit reactor 110 and a reaction product from this contacting is collected in the separator 120. The reaction products include the organic phase 120a, the aqueous phase 120b, and, optionally, an emulsion layer therebetween. The organic phase 120a includes cannabinoids and an organic solvent from the cannabinoid containing compositions. The aqueous phase 120b includes the aqueous solution and at least one impurity from the cannabinoid containing composition, which may be as described above.

In step 208, the organic phase 120a (and optionally an emulsion layer) is directed back into the inlet 110a of the first conduit reactor 110. In step 210, a second aqueous solution is supplied to an inlet 110a of the first conduit reactor 110 from one or more of the first aqueous supply 104, the second aqueous supply 106, and the third aqueous supply 108 via lines 105, 107, and 109, respectively. The second aqueous solution may be the same or different from the first aqueous solution. In some embodiments, the first aqueous solution is an acidic solution from the first aqueous supply 104 or a basic solution from the second aqueous supply 106 and the second aqueous solution is water or brine from the third aqueous supply 108. In a step 212, the second aqueous solution and the organic phase 120a are contacted with one another in the first conduit reactor 110 and reaction products from the contact are collected in the separator 120. The reaction products include a second organic phase including cannabinoids and an organic solvent from the organic phase 120a and a second aqueous phase that includes the second aqueous solution and at least one impurity from the organic phase 120a. The at least one impurity may be as described above. In step 212, the second organic phase is separately removed from the separator 120 and directed via line 113 to the cannabinoid oil tank 130.

In some embodiments, the method 200 may include additional iterations of steps 208 and 210. That is, the method 200 may include a third cycle through the first conduit reactor 110, wherein the second organic phase is reintroduced into the first conduit reactor 110 with a third aqueous solution. In some embodiments, the method 200 include first and second runs through the first conduit reactor 110 with an acidic solution from first aqueous supply 104 and a basic solution from the second aqueous supply 106 (in either order) and then a third and final run with water or brine from the third aqueous supply 108. In some embodiments, the method 200 include 4 or more runs through the first conduit contactor.

In some embodiments, the method 200 includes one or more steps of removing the aqueous phase 120b via lines 121 from the separator 120 to the effluent tank 122. The method 200 may also include steps of recycling, remediating, or discarding contents of the effluent tank 122.

In any of embodiments, the rate of introducing the organic phase (either from the separator 120 or from the cannabinoid supply 102) and the aqueous solution from supplies 104, 106, and/or 108 may be adjusted as needed. In some embodiments, a ratio of a rate (gallons/hours) of organic phase introduction to a rate (gallons/hour) of aqueous solution is 10:1 to 1:100, 1:1 to 1:50, 1:1 to 1:20, or 1:1 to 1:10.

The method 200 may be run at any appropriate temperature, i.e., the system 100 or parts thereof may be maintained at an appropriate temperature. For example, the temperature may be in the range of 1° C. to 100° C. Elevated temperatures may decrease the extraction time, but the temperature should not be so high as to degrade (e.g., decarboxylate) components of the organic phase or cause the liquids to boil. Moreover, the method 200 may be run at any appropriate pressure. For example, the pressure may be in the range of 0 to 300 psi. Temperature and/or pressure sensors may be included at any locations within the system 100 to monitor the reaction temperature and/or pressure. In some embodiments, any vessel (e.g., in supplies 102, 104, 106, 108, separator 120, or tanks 122, 130) may include a liquid level sensor, which may be in communication with the controller. In some embodiments, a liquid level sensor may be included in the separator 120 and the liquid level sensor may be configured to determine a level of the aqueous phase 120b and/or the organic phase 120a or an interface position therebetween.

In some embodiments, the system 100 includes one or more pH sensors. For example, any of lines 103, 105, 107, 109, 111, 121, and/or 113 may include a pH sensor. In some embodiments, the separator 120 include a pH sensor. In some embodiments, the separator 120 includes a pH sensor for the organic phase 120a, for the aqueous phase 120b, and/or for an emulsion layer. In some embodiments, supplies 102, 104, 106, and/or 108 includes a pH sensor. In some embodiments, cannabinoid oil tank 130 and/or effluent tank 122 includes a pH sensor.

In some embodiments, the temperature sensors, pressure sensors, and/or pH sensors communicate temperature, pressure, and pH data, respectively, to a controller. In response to the data received from the sensors, the controller may be configured to, e.g., control any of the valves 102a, 104a, 106a, 108a, 111a, 121a in the system 100 to increase, decrease, or cease flow through any of the lines 103, 105, 107, 109, 111, 113, 121. In some embodiments, the system 100 may include one or more heaters that are controllable by the controller in response to the data received from the sensors. In some embodiments, the controller may include a control panel and user interface for monitoring and control of the system 100. In some embodiments, the system 100 is automated on the basis of pH. For example, the system 100 may run the organic phase through one or more cycles in the first conduit contactor 110 and continue until a threshold pH is measured in the organic phase 120a of the separator, at which point the supply 104, 106, 108 is changed or the organic phase 120a is directed to the cannabinoid oil tank 130. In some embodiments, the method 200 is continuous wherein a run or cycle may be considered complete when the entire volume of organic phase has passed through the first conduit reactor 110. In some embodiments, the aqueous solution is continually supplied by at least one of the supplies 104, 106, 108. In such embodiments, the valves 104a, 106a, and 108a may be selectively opened and closed to alter the composition of the aqueous solution in response to data received by the controller from the sensors. For example, the valve 104a may be opened while valves 106a and 108 are closed in order to supply an acidic solution to the first conduit reactor 110. Then, once a threshold pH is measured in the reaction products, the valve 104a may be closed and the valve 106a or the valve 108a may be opened. In some embodiments, the threshold pH is not a set number but a degree of change in pH from a previous point in the reaction. For example, the threshold pH may occur when two consecutive pH measurements differ by less than 1, less than less than 0.2, less than 0.1, or less than 0.05, or do not differ. The two consecutive pH measurements may be separated by a set period of time dictated by the rate at which materials are introduced into the first conduit reactor 110. For example, pH measurements may be taken every 10 seconds, 30 seconds, 60 seconds, 120 second, 5 minutes, 10 minutes, 15 minutes, 30 minutes, or 60 minutes. In some embodiments, the pH measurements are continuously taken and the threshold pH is met when the pH changes by less than by less than 1, less than 0.5, less than less than 0.1, or less than 0.05, or does not change over a period of 10 seconds, 30 seconds, seconds, 120 second, 5 minutes, 10 minutes, 15 minutes, 30 minutes, or 60 minutes.

In some embodiments, an organic phase may be simultaneously introduced into the first conduit reactor from both the cannabinoid supply 102 and the separator 120. In such embodiments, the valve 102a may be closed after a set period of time or after a threshold condition is measured in the reaction products (e.g., in the separator 120, effluent tank 122, line 111, and/or line 121). The closing of valve 102a may be automated using the controller. The organic phase 120a may then continue to be recycled through the first conduit reactor 110 or all or a portion thereof may be directed to the cannabinoid oil tank 130.

In one or more embodiments, the cannabinoid containing composition is a crude cannabinoid extract and the crude cannabinoid extract only undergoes a single cycle through the first conduit reactor 110. In some embodiments, the crude cannabinoid extract is simultaneously introduced into the first conduit reactor with a basic solution having a pH of 8.5-9.5 and a temperature of the system 100 is maintained at about 10-40° C., about 20-35° C., or about 30° C.

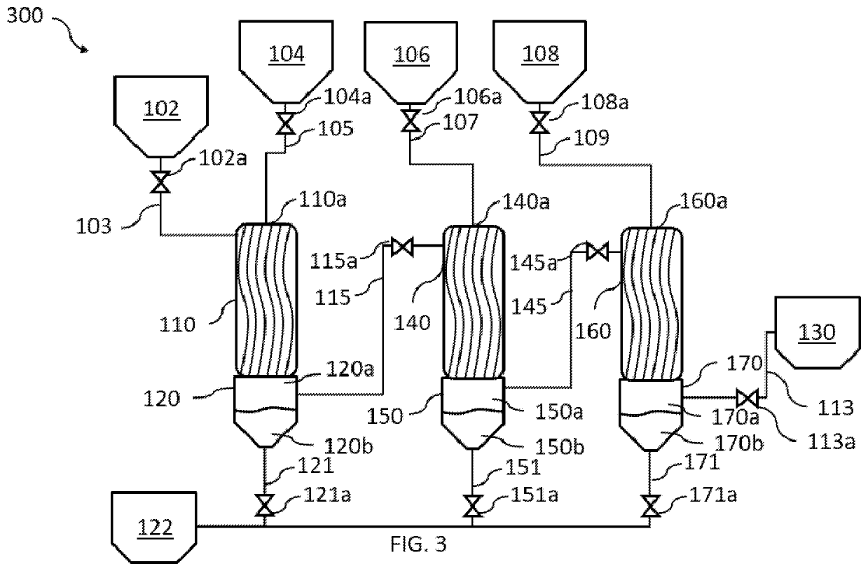

Turning to FIG. 3, another system 300 is depicted. Like reference numbers from FIG. 1 refer to like elements unless noted otherwise. In the system 300, rather than recycling the organic phase 120a back into the first conduit reactor 110, a second conduit reactor 140 and third conduit reactor 160 are used to further process the organic phase 120a. In particular, the first conduit reactor 110 is supplied with a cannabinoid containing composition from the cannabinoid supply 102 via line 103 and simultaneously supplied with an aqueous solution from the first aqueous supply 104 via line 105. In any embodiments, the first, second, and/or third supplies 104, 106, 108 may be rearranged, run counter current, duplicated, or omitted. For example, the first aqueous supply 104 may be switched with the second aqueous supply 106. As another example, the first aqueous supply 104 may be connected to both the first conduit reactor 110 and the second conduit reactor 140 and the second aqueous supply 106 may be omitted, or vice versa. Additionally, although three reactors 110, 140, 160 are shown, the system 300 may include two or four or more reactors connected in series with each being connected to at least one aqueous supply 104, 106, 108.

After the cannabinoid containing composition and the aqueous solution from the first aqueous supply 104 have been contacted in the first conduit reactor, the reaction products are collected in the separator 120. An organic phase 120a (and optionally an emulsion layer) is then directed via line 115, which may include a valve 115a to control flow therethrough, to an inlet 140a of the second conduit reactor 140. The second conduit reactor 140 is simultaneously supplied at the inlet 140a with an aqueous solution from the second aqueous supply 106 via line 107. The organic phase 120a is contacted with the aqueous solution in the second conduit reactor 140 and the reaction products are collected in a second separator 150. The reaction products may include an organic phase 150a, an aqueous phase 150b, and optionally an emulsion layer therebetween. The organic phase 150a include cannabinoids from the organic phase 120a and the aqueous phase 150b include the aqueous solution from the second aqueous supply 106 and at least one impurity from the organic phase 120a, which may be as described above regarding the impurities in the cannabinoid containing composition. The aqueous phase 150b (and optional the emulsion layer) may be directed via line 151, which may include a valve 151a, to the effluent tank 122. The organic phase 150a (and optional the emulsion layer) is directed via line 145, which may include a valve 145a, to an inlet 160a of the third conduit reactor 160.

The third conduit reactor 160 is simultaneously supplied at the inlet 160a with an aqueous solution from the third aqueous supply 108 via line 109. The organic phase 150a is contacted with the aqueous solution in the third conduit reactor 160 and the reaction products are collected in a third separator 170. The reaction products may include an organic phase 170a and an aqueous phase 170b. The organic phase 170a include cannabinoids from the organic phase 150a and the aqueous phase 170b include the aqueous solution from the third aqueous supply 108 and at least one impurity from the organic phase 150a, which may be as described above with reference to the cannabinoid containing composition. The aqueous phase 170b may be directed via line 171, which may include a valve 171a, to the effluent tank 122. The organic phase 170a is directed via line 113, which may include a valve 113a, to the cannabinoid oil tank 130.

Any of the first conduit reactor 110, second conduit reactor 140, and third conduit reactor 160 may include a line connecting the respective separator 120, 150, 170 back to the respective reactor 110, 140, 160. That is, the organic phase 120a may be recycled into the first conduit reactor 110, the organic phase 150a may be recycled into the second conduit reactor 140, and/or the organic phase 170a may be recycled into the third conduit reactor 160. Any recycling lines may include valves for controlling flow therethrough. In some embodiments, the third conduit reactor 160 (or a final reactor when using two or four or more reactors) is supplied with water or brine by the third aqueous supply 108. That is, in some embodiments, the final wash is with water or brine.

The system 300 may include temperature sensors, pressure sensors, liquid level sensors, and/or pH sensors as described above. The system 300 may also include the controller described above, which may be configured to control, e.g., valves 102a, 104a, 106a, 108a, 121a, 115a, 151a, 145a, 171a, and/or 113a. In some embodiments, the system 300 is automated via the controller and sensors in the manner described above.

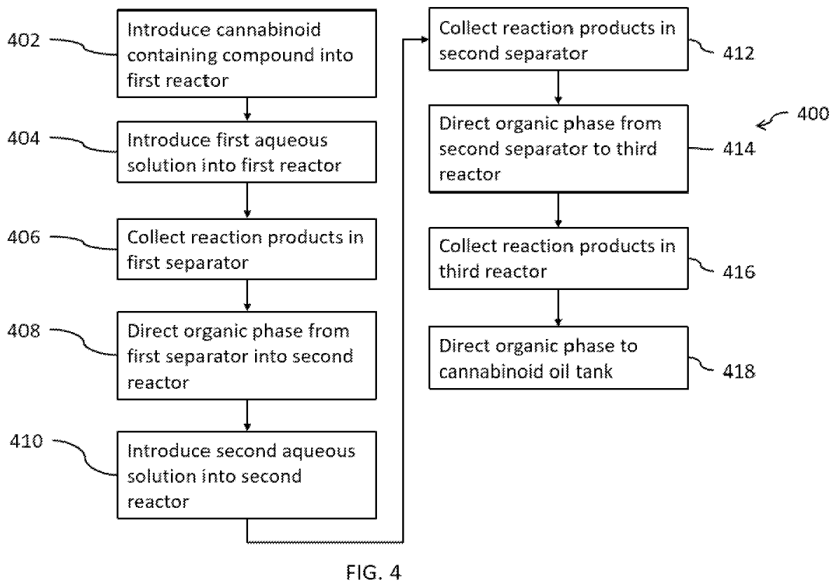

Turning to FIG. 4, a method 400 of operating the system 300 is depicted. In step 402, the cannabinoid containing composition is supplied into an inlet 110a of the first conduit reactor 110 via line 103. In step 404, which may be conducted simultaneously with step 402, a first aqueous solution is supplied to the inlet 110a of the first conduit reactor 110 from the first aqueous supply 104 via line 105. In step 406, the cannabinoid containing composition and aqueous solution are contacted with one another in the first conduit reactor 110 and a reaction product from this contacting is collected in the separator 120. The reaction products include the organic phase 120a, the aqueous phase 120b, and, optionally, an emulsion layer therebetween. The organic phase 120a includes cannabinoids and an organic solvent from the cannabinoid containing compositions. The aqueous phase 120b includes the aqueous solution and at least one impurity from the cannabinoid containing composition, which may be as described above.

In step 408, the organic phase 120a (and optionally an emulsion layer) is directed via line 115 to the inlet 140a of the second conduit reactor 140. In step 410, a second aqueous solution is supplied to the inlet 140a of the second conduit reactor 140 from the second aqueous supply 106 via line 107. The second aqueous solution may be the same or different from the first aqueous solution. In a step 412, the second aqueous solution and the organic phase 120a are contacted with one another in the second conduit reactor 140 and reaction products from the contact are collected in the separator 150. The reaction products include a second organic phase 150a including cannabinoids and an organic solvent from the organic phase 120a and a second aqueous phase 150b that includes the second aqueous solution and at least one impurity from the organic phase 120a, which may be as described above with reference to the cannabinoid containing composition. In step 412, the second organic phase 150a is directed via line 145 to the inlet 160a of the third conduit reactor 160. In step 414, a third aqueous solution is supplied to the inlet 160a of the third conduit reactor 160 from the third aqueous supply 108 via line 109. The third aqueous solution may be the same or different from the first or second aqueous solution. In some embodiments, the third aqueous solution is water or brine. In step 416, the third aqueous solution and the second organic phase 150a are contacted with one another in the third conduit reactor 160 and reaction products from the contact are collected in the separator 170. The reaction products include a third organic phase 170a including cannabinoids and an organic solvent from the second organic phase 150a and a third aqueous phase 170b that includes the third aqueous solution and at least one impurity from the organic phase 150a, which may be as described above with reference to the cannabinoid containing composition. In step 418, the organic phase 170a is directed via line 113 to the cannabinoid oil tank 130.

In some embodiments, the method 400 includes steps of removing the aqueous phase 120b, 150b, 170b via lines 121, 151, 171, respectively, from the separator 120, 150, 170 to the effluent tank 122. The method 400 may also include steps of recycling, remediating, or discarding contents of the effluent tank 122.

In some embodiments, the method 400 may recycling steps of reintroducing the organic phase 120a, 150a, 170a into the first, second, or third conduit reactor 110, 140, 160. That is, the method 400 may include reintroducing the organic phase 120a into the first conduit reactor 110, reintroducing the organic phase 150a into the second conduit reactor 140, and/or reintroducing the organic phase 170a into the third conduit reactor 160. In some embodiments, the system 300 includes two or four or more reactors and the method 400 may be adjust accordingly to include fewer or additional steps of introducing an organic phase and an aqueous phase into the respective reactors. Temperature, pressure, and flow rate condition of the method 400 may be as described above.

In any of the above embodiments, pumps may be included as needed to assist in flow of the materials through the system 100, 300. Agitators may also be included in any vessel (e.g., in supplies 102, 104, 106, 108, or tanks 122, 130). Agitators may include, but are not limited to, mechanical mixers or stirrers or air agitators (bubblers).

In some embodiments, the systems 100, 300 and methods 200, 400 described herein may be used to convert cannabinoids (e.g., CBD to delta 8 THC) and/or enrich synthetically derived acidic cannabinoid products from their natural, neutral cannabinoid precursors (e.g., CBDA from CBD). Additionally, the methods described herein can be used to remove catalysts used in these conversions (e.g., zinc chloride and/or pTSA) from the desired final products (e.g., delta 8 THC) via a series of washes. For example, the cannabinoid containing composition may include a synthetic cannabinoid having a mixture of a precursor and a synthesized cannabinoid. In some embodiments, the synthetic cannabinoid may be dissolved in an organic solvent such as those described above (e.g., heptane) in order to form the cannabinoid containing composition used herein. In some embodiments, the synthetic cannabinoid includes a mixture of precursor and synthesized cannabinoid in a weight ratio of 2:1 to 1:5, 2:1 to 1:3, 1:1 to 1:2, or about 1:1. In some embodiments, the system 100, 300 converts at least 20 wt %, at least 40 wt %, at least 50 wt %, at least 60 wt %, at least 70 wt %, at least 80 wt %, at least 90 wt %, or about 100 wt % of the precursor into synthesized cannabinoid.

In some embodiments, the precursor includes terpenes and the synthesized cannabinoid includes a neutral cannabinoid or the precursor includes a cannabinoid and the synthesized cannabinoid is a different cannabinoid. In such embodiments, the system 100, 300 may be configured to contact the synthetic cannabinoid with a solution containing the desired catalysts and/or solutions described as extremely acidic or basic (e.g., pH of <2 or >13).

In some embodiments, the precursor is a neutral cannabinoid and the synthesized cannabinoid is an acid form of the neutral cannabinoid. For example, the precursor may be CBD and the synthesized cannabinoid may be CBDA. In such embodiments, the system 100, 300 may be configured to contact the synthetic cannabinoid with an acidic solution having a pH of less than 6, less than 5, less than 4, 2 to 6, 3 to 6, or 3.5 to 6 in a conduit reactor to convert precursor into synthesized cannabinoid.

According to embodiments of the present disclosure, the system and method described herein may facilitate a reaction between an organic phase containing cannabinoids and an aqueous phase used to extract contaminants from the organic phase without the need for increased pressure and temperature, which may cause unwanted reactions (such as decarboxylation or conversion of cannabinoids or related compounds). Additionally, the system and method described herein are tunable to accommodate the wide variation in compositions that naturally result from hemp extractions. For example, by using a series of washes with varying aqueous solutions, the system and method can remove undesirable contaminants in a stepwise manner to yield a clean cannabinoid oil.

Although various embodiments have been shown and described, the disclosure is not limited to such embodiments and will be understood to include all modifications and variations as would be apparent to one of ordinary skill in the art. Therefore, it should be understood that the disclosure is not intended to be limited to the particular forms disclosed; rather, the intention is to cover all modifications, equivalents, and alternatives falling within the spirit and scope of the disclosure as defined by the appended claims.

What is claimed is:

1. A method comprising:
simultaneously introducing a cannabinoid containing composition and a first aqueous solution into a first end of a first conduit reactor comprising a plurality of fibers disposed therein;
collecting first reaction products in a first separator disposed opposite the first end;
wherein the cannabinoid containing composition is immiscible with water and comprises a cannabinoid, a first impurity, and a second impurity, where the first and second impurities may be of the same kind or of different kinds; and
wherein the first reaction products comprise a first organic phase comprising the cannabinoid and the second impurity, and a first aqueous phase comprising the first aqueous solution and the first impurity;
simultaneously introducing the first organic phase and a second aqueous solution into the first end of the first conduit reactor or into a first end of a second conduit reactor comprising a plurality of fibers disposed therein, wherein the second aqueous solution may be of the same kind or of a different kind than the first aqueous solution;
collecting second reaction products in the first separator or in a second separator disposed opposite the first end of the second conduit reactor;
wherein the second reaction products comprise a second organic phase comprising the cannabinoid and a second aqueous phase comprising the second aqueous solution and the second impurity; and
separately removing the second organic phase.

2. The method of claim 1, wherein the organic phase and the second aqueous solution are simultaneously introduced into the first end of the first conduit reactor; and
wherein the method further comprises:
measuring a first pH of the first aqueous solution;
measuring a second pH of the second aqueous solution;
comparing the first pH and second pH;
if a difference between the first pH and second pH is greater than 0.5, reintroducing the second organic phase into the first end of the first conduit reactor with additional second aqueous solution.

3. The method of claim 1, wherein the first aqueous solution is an acidic solution or a basic solution and the second aqueous solution is water or brine.

4. The method of claim 1, wherein the cannabinoid containing composition further comprises a third impurity that may be of the same kind or of a different kind than the first impurity or the second impurity;
wherein the second organic phase comprises the third impurity; and
wherein the method further comprises:
simultaneously introducing the second organic phase and a third aqueous solution into the first end of the first conduit reactor, the first end of the second conduit reactor, or a first end of a third conduit reactor comprising a plurality of fibers disposed therein, wherein the third aqueous solution may be of the same kind or of a different kind than the first and/or second aqueous solution;
collecting third reaction products in the first separator, the second separator, or a third separator disposed opposite the first end of the third conduit reactor;
wherein the third reaction products comprise a third organic phase comprising the cannabinoid and a third aqueous phase comprising the third aqueous solution and the third impurity.

5. The method of claim 4, wherein the first aqueous solution is one of an acidic solution or a basic solution, the second aqueous solution is the other of the acidic solution or the basic solution, and the third aqueous solution is water or brine.

6. The method of claim 4, wherein the first aqueous solution is an acidic solution, the second aqueous solution is a basic solution, and the third aqueous solution is water or brine.

7. The method of claim 1, wherein the first impurity comprises free fatty acids (FFAs), pesticides, gums, phospholipids, colorants or pigments, odor causing substances, acidic cannabinoids, sugars, cellulose, hemicellulose, chlorophyl, or combinations thereof.

8. The method of claim 3, wherein simultaneously introducing the first organic phase and the second aqueous solution comprises introducing into the first end of the first conduit reactor.

9. The method of claim 5, wherein simultaneously introducing the first organic phase and the second aqueous solution comprises introducing into the first end of the first conduit reactor and wherein simultaneously introducing the second organic phase and the third aqueous solution comprises introducing into the first end of the first conduit reactor.

10. The method of claim 3, wherein simultaneously introducing the first organic phase and the second aqueous solution comprises introducing into the first end of the second conduit reactor.

11. The method of claim 5, wherein simultaneously introducing the first organic phase and the second aqueous solution comprises introducing into the first end of the second conduit reactor and wherein simultaneously introducing the second organic phase and the third aqueous solution comprises introducing into the first end of the third conduit reactor.

12. The method of claim 1, wherein the cannabinoid containing composition comprises a first cannabinoid or a terpene;
wherein the cannabinoid containing composition or the first aqueous solution comprises a catalyst capable of converting the first cannabinoid or the terpene into a second cannabinoid that is different than the first cannabinoid and the terpene; and
wherein the first aqueous solution has a pH of less than 2 or greater than 13.

13. The method of claim 12, wherein the cannabinoid containing composition comprises cannabidiol (CBD) and the second cannabinoid is tetrahydrocannabinol (THC).

14. The method of claim 1, wherein the first aqueous solution or the second aqueous solution comprises a phase transfer catalyst or a chelating agent.

15. A system comprising:
a cannabinoid supply comprising a cannabinoid containing composition disposed therein, wherein the cannabinoid containing composition comprises a cannabinoid, a first impurity, and a second impurity;
a first aqueous supply comprising a first aqueous solution disposed therein;
a second aqueous supply comprising a second aqueous solution disposed therein, wherein the second aqueous solution has a different pH than the first aqueous solution;
a conduit reactor comprising a plurality of fibers disposed therein and a first end coupled to the cannabinoid supply, the first aqueous supply, and the second aqueous supply;
a separator positioned opposite the first end and configured to accept reaction products from the cannabinoid containing composition and the first aqueous solution or the second aqueous, wherein said reaction products include an organic phase comprising the cannabinoid and an aqueous phase comprising the first aqueous solution and the first impurity or the second aqueous solution and the second impurity;
a sensor positioned within the separator and configured to measure a pH of the aqueous phase; and
a recirculation line comprising a pump and configured to direct the organic phase from the separator to the first end of the conduit reactor.

16. The system of claim 15, further comprising:
a first valve positioned between the cannabinoid supply and the first end of the conduit reactor;
a second valve positioned between the first aqueous supply and the first end of the conduit reactor;
a third valve positioned between the second aqueous supply and the first end of the conduit reactor;
a controller configured to receive pH data from the sensor and to operate the pump, the first valve, the second valve, and the third valve in response to said pH data.

17. The system of claim 16, further comprising
a third aqueous supply comprising a third aqueous solution disposed therein, wherein the third aqueous solution has a different pH that the first aqueous solution and the second aqueous solution; and
a fourth valve positioned between the third aqueous supply and the first end of the conduit reactor;
wherein the controller is further configured to operate the fourth valve.

18. The system of claim 17, wherein the first aqueous solution is acidic, the second aqueous solution is basic, and the third aqueous solution is water or a brine.

19. The system of claim 16, wherein the controller is configured to:
(i) open the first valve and the second valve for a first set period of time to simultaneously introduce the cannabinoid containing composition and the first aqueous solution into the first end of the conduit reactor;
(ii) close the first valve and the second valve and open the third valve and operate the pump for a second set period of time to simultaneously introduce the organic phase from the separator and the second aqueous solution into the first end of the conduit reactor; and
(iii) repeat step (ii) until a difference in pH values between subsequent steps falls below a threshold value.

20. The system of claim 15, wherein the cannabinoid supply, the first aqueous supply, or the second aqueous supply comprises an agitator.
